(19) United States
(12) Patent Application Publication
(10) Pub. No.: US 2021/0166808 A1
(43) Pub. Date: Jun. 3, 2021

(54) SYSTEM AND METHOD FOR TRANSFER OF RESIDENTS IN RESIDENTIAL FACILITIES

(71) Applicant: Kimberly Geis, Napa, CA (US)

(72) Inventor: Kimberly Geis, Napa, CA (US)

(21) Appl. No.: 17/177,990

(22) Filed: Feb. 17, 2021

Related U.S. Application Data
(63) Continuation of application No. 16/664,807, filed on Oct. 26, 2019.

(51) Int. Cl. G16H 40/20; G06Q 50/16; G06Q 10/06; G06Q 50/26; G06Q 10/02; G06Q 10/10; G06F 3/0486; G06F 3/0482

(52) U.S. Cl. CPC G16H 40/20; G06Q 50/163; G06Q 10/063118; G06Q 50/26; G16H 10/60; G06Q 10/103; G06F 3/0486; G06F 3/0482; G06Q 10/02

(57) ABSTRACT

An interactive user interface (UI) accessible on a computer network through a connected computing device is provided, the UI enabling request for transfer and initiation thereof of a resident in a source residential facility connected to a network to a destination residential facility connected to the network. The request for transfer is, in one aspect, accomplished by copy and paste or drag and drop of a link representing a resident into a bed, room, or other space of a facility floor plan of the destination facility.

### CROSS-REFERENCE TO RELATED DOCUMENTS

The present invention is a continuation in part (CIP) of a U.S. patent application Ser. No. 16/664,807 entitled Systems and Methods for Ensuring Facility Compliance filed on Oct. 26, 2019, disclosure of which may be included herein at least by reference.

### BACKGROUND OF THE INVENTION

### 1. Field of the Invention

The present invention is in the field of assisted living facilities including management thereof and pertains particularly to methods and apparatus for relocation of one or more persons among assisted living facilities during a crisis or during a time of need.

### 2. Discussion of the State of the Art

At the time of this writing, the Community Care Licensing Division in California has licensed over 72,400 care facilities having a capacity to serve at least one and one quarter of a million Californians. Of these facilities, eight-thousand plus are residential care facilities for servicing elderly tenants in a residential or urban living environment. The number of facilities has a combined capacity to serve at least two hundred-thousand tenants in need of care or assisted living. Of the eight-thousand facilities certified by Resource for Continuing Education (RFCE), approximately six-thousand facilities may include six beds or fewer for tenants who reside full-time at these facilities. Such residential care facilities are unique and are typically funded by the residents of those facilities.

The inventor is aware of a system for ensuring facility compliance, and this system is referenced in this specification in the related documents section as U.S. patent application Ser. No. 16/664,807 entitled Systems and Methods for Ensuring Facility Compliance filed on Aug. 26, 2019. The system known to the inventor includes a centralized a filing system for all required residential facility or assisted living facility documents.

The system enables staff to chart Activities of Daily Living (AOL), Instrumental Activities of Daily Living (IADLs), simple care notes, and medication passes. Facility administrators, licensees, and state licensing agents can perform compliance reviews including compliance audits on documents associated with facilities from remote office locations over a network. Each facility is digitally represented, and updated data is accessible to those on the network with authority to access the data. The system known to the inventor may be turn-keyed for other types of facilities that provide care or services including home care organizations, child care centers, infant care centers, after school centers, family care homes, group homes, small family homes, foster homes, or rehabilitation facilities.

In times of emergency, it may be necessary for authorities to issue mandatory evacuations of large regions due to fires, weather events like hurricanes, virus pandemics, and so on. In any state, these facilities may be scattered throughout a region, some urban, some suburban, and some rural. It has occurred to the inventor that ad-hoc networks and warning systems often are not suitable for relocating a tenant with certain medical requirements from a home or facility under temporary or longer-term emergency evacuation orders.

Therefore, what is clearly needed is a system and method for finding and reserving emergency relocation for one or more persons among existing assisted living facilities.

### BRIEF SUMMARY OF THE INVENTION

According to at least one embodiment of the present invention, an Interactive User Interface (UI) accessible on a computer network through a connected computing device is provided. The UI enables users to request for transfer and initiation of transfer of a resident in a source residential facility connected to the network to a destination residential facility connected to the network. The UI enabled by coded instruction residing on a non-transitory medium coupled to a server connected to the network; the server coupled to at least one data repository, and the coded instruction commanding the server to (a) receive data from residential facilities connected to the network, to (b) store the data received in (a) in updated state in the at least one data repository, to (c) serve at least one digital instance of the UI to at least one authority having charge over at least one of the residential facilities upon request of the at least one authority, to (d) serve at least one digital map overlaid with interactive indicia geographically and texturally identifying the connected facilities to the at least one authority of (c) upon request, to (e) receive a request for transfer of one or more residents from the one or more connected source facilities to one or more connected destination facilities, to (f) run a comparative algorithm between data stored for the one or more source facilities and data stored for one or more of the destination facilities of the request received in (e), to (g) alert based on the data comparison of (f) or do not alert the requesting authority or authorities of a potential conflict with the desired goal or goals of the requested transfer of (e), to (h) serve notification of transfer request, transfer schedule, and required documents to one or more destination authorities for review and response, to (i) provide destination authority(s) acceptance as a response, confirm via server notification to the requesting authority or authorities, process successful transfer of the one or more residents or (upon authority) rejection as a response, reject via server notification to the requesting authority or authorities transfer of the one or more residents, and to (j) update map meta-data associated with the interactive indicia overlaid on the digital map.

In one embodiment, the network is the Internet network and any connected sub networks. In a preferred embodiment, the received data (a) is uploaded from the facilities over the Internet. In one embodiment, the residential facilities include permanent-resident residential facilities, emergency facilities, and temporary-resident residential facilities.

In one embodiment, the residential facilities include residential care homes defined by converted houses, and residential care facilities defined by converted commercial buildings. In a preferred embodiment, the UI includes multiple interactive options for linking to and viewing categorically diverse data sets associated with any type of residential facility. In this embodiment, data about each facility's physical capacities, staff, residents, emergency preparedness, and safety planning.

In one embodiment, the connected device is a mobile telephone device having connection to the network in a physical location outside of a facility. In one embodiment, data received in (a) includes a list of the number of current residents at each facility and the current and scheduled number of vacancies, if any, at each facility. In one embodiment, the data received in (a) includes one or more digital floor plans overlaid with interactive indicia geographically and texturally identifying at least occupied, non-occupied and/or otherwise vacant living spaces.

In a preferred embodiment, the interactive indicia geographically and texturally identifying the connected facilities in the digital map of (d) contain links to the parent facility data when selected by the one or more authorities in the digital map, the multiple data options in the UI automatically link to those data sets upon selection. In this embodiment, at least one of the multiple data options enables view and interaction with the one or more digital floor plans of claim 11.

In a preferred embodiment, interaction with the one or more digital floor plans of claim 11 include computer-aided copying and pasting of one or more objects representing residents needing transfer onto the vacant rooms or beds of the one or more floor plans, or include computer-aided dragging and dropping of one or more objects representing residents needing transfer onto the vacant rooms or beds of the one or more floor plans. In this embodiment, the computer interaction methods enable copy and paste and/or drag and drop as stated in claim 14 comprising the one or more requests for transfer in (e).

### BRIEF DESCRIPTION OF THE SEVERAL VIEWS OF THE DRAWINGS

FIG. 1 illustrates the modules of the record system software.

FIG. 2 illustrates the administrator dashboard module.

FIG. 3 illustrates the resident module.

FIG. 4 illustrates the medications module.

FIG. 5 illustrates the caregiver's module.

FIG. 6 illustrates the staff module.

FIG. 7 illustrates the facility module.

FIG. 8 illustrates the admin module.

FIG. 9 illustrates Licensed Program Analyst (LPA).

FIG. 10 is a network diagram depicting an emergency patient transfer (EPT) network according to an embodiment of the present invention.

FIG. 11 is a screen shot of an ERT dashboard panel of the Software (SW) of FIG. 1 depicting an emergency disaster plan.

FIG. 12 is a screen shot of an ERT dashboard panel of the SW of FIG. 1 depicting a state map of facilities registered on the network.

FIG. 13 is a screen shot of an ERT dashboard panel of the SW of FIG. 1 depicting a selected region of the state map of FIG. 12.

FIG. 14 is a screen shot of an ERT dashboard panel of the SW of FIG. 1 depicting the selected region of FIG. 13 highlighting available destination facilities.

FIG. 15 is a screen shot of an ERT dashboard panel of the SW of FIG. 1 depicting a facility diagram.

FIG. 16 is a screen shot of an ERT dashboard panel of the SW of FIG. 1 depicting the selected region of FIG. 12 highlighting temporary facilities and permanent facilities having vacancies.

FIG. 17 is a screen shot of an ERT dashboard panel of the SW of FIG. 1 depicting a source facility floor plan and a destination facility floor plan side by side.

FIG. 18 is a screen shot of an ERT dashboard panel of the SW of FIG. 1 depicting the facility floor plans of FIG. 17 with an alert overlay noting a resident transfer conflict with the destination facility.

FIG. 19 is a process flow chart depicting steps for requesting at least one resident transfer using the ERT system of the invention.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention.

Reference will now be made in detail to selected embodiments of the present disclosure in conjunction with accompanying figures. The embodiments described herein are not intended to limit the scope of the disclosure, and the present disclosure should not be construed as limited to the embodiments described. This disclosure may be embodied in different forms without departing from the scope and spirit of the disclosure. The accompanying figures are intended and provided to illustrate embodiments of the disclosure described below and are not necessarily drawn to scale. In the drawings, like numbers refer to like elements throughout, and thicknesses and dimensions of some components may be exaggerated for providing better clarity and ease of understanding.

It should be noted that the terms “first”, “second”, and the like, herein do not denote any order, ranking, quantity, or importance, but rather are used to distinguish one element from another. Further, the terms “a” and “an” herein do not denote a limitation of quantity, but rather denote the presence of at least one of the referenced items. All illustrations of the drawings are for the purpose of describing selected versions of the present invention and are not intended to limit the scope of the present invention.

The records system software of the present invention meets the daily needs of the administrator and staff caring for residents. This records system software keeps up to the minute at fingertips information. The records system of the present invention based on a monthly subscription. The monthly subscription of the record system software will streamline all required documents for the Residential Care Facility for the Elderly (RCFE), this includes all the documents needed for staff, resident, facility, and medications. The records system software digitizes the process of operating a California State licensed facility such as RCFE and ARF. This records system software helps the facility owner remain in compliance with all CCLD DSS (Community Care Licensing Division Department of Social Services) title regulations.

The records system software of the present invention will be targeting all State licensed Facilities in California, there are approximately 7500 licensed facilities in California with 500 pending licensing statuses. There are 5500 6 bed facilities targeted that have not converted to an electronic facility record, it is hard for them to operate as the owner/operator must do assessments, discharges, hospice care, prescription calling and pick up working shifts caring for elderly. Every facility has an administrator, this administrator is responsible for all facility, staff, residents, and medication documents and for a 6-bed facility it over 133 required documents.

The user will upload all required document forms and the program has default expiration dates, as a form becomes expired and requires review, an alert will be sent to dashboard. The program focuses on residents, staff and facility required documents as well as medication pass and pour. Beside the records system software of the invention, there is a process where the user fills out all questions and uploads all required documents into the system. The program is made up of coding that runs the program.

The software of the present invention is designed for both the individual care homes and the compliance agencies. The Software allows the RCFE's portal to manage the numerous documents that they are required to maintain to remain in good standing with the State of California as well as assist them with day-to-day operations. The software is a secure website that was custom built using industry leading technologies for flexibility and security with a focus on a simple user-friendly interface. The software is secure to handle both HIPPA and PII (Personal Identifying Information, see CA bill number SB 1386) data. This system also adds the benefit that all the documentation will be stored electronically providing business and records continuity in the event of a fire or other disaster.

The software of the present invention provides facility information stored electronically in the system and can be viewed by the care homeowner, administrator, or state officials from any location using a secure log-in. By using the present invention software, the facility compliance is automatically analyzed and documented based on the care home's classifications. The items that are out of compliance are highlighted on the administrator's dashboard. The administrator can then work to resolve the compliance issue(s).

Resident information is readily available to the staff in the facility. The system will keep track of everything related to each resident and provides a recap of their care for inquiries from family, doctors, etc.

To use the records system software of the present invention, a user will sign up, then the user will go to the payment page to pay the fee of 69.00 per month through Pay pal button then receive welcome email within 24 hrs.

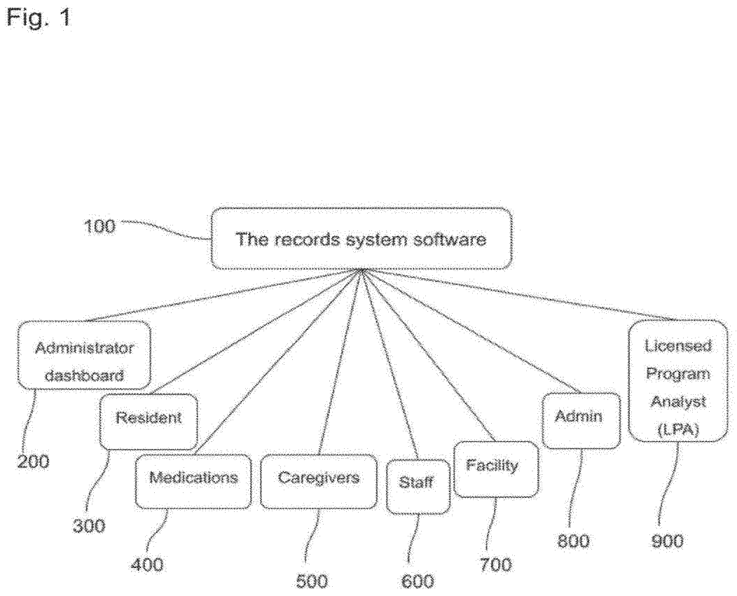

Referring to FIG. 1, after a user log-in, the user will see the modules of administrator dashboard 200, resident 300, medications 400, caregivers 500, staff 600, facility 700, admin 800, and Licensed Program Analyst (LPA) 900. In the following parts, we will explain every module in detail:

### Administrator Dashboard Module

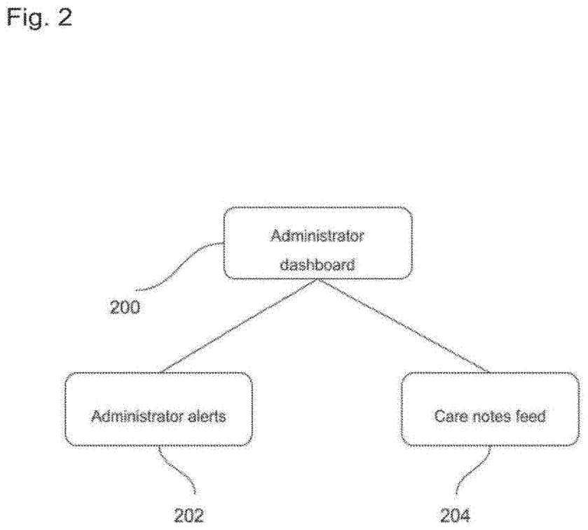

Referring to FIG. 2, the administrator dashboard module 200 provides all the relevant information for administrators to review/address prioritized by urgency and/or compliance risk. The dashboard module 200 contains two sections, administrator alerts 202 and care notes feed 204. The notes include any expired documents will be marked as urgent.

### Resident Module

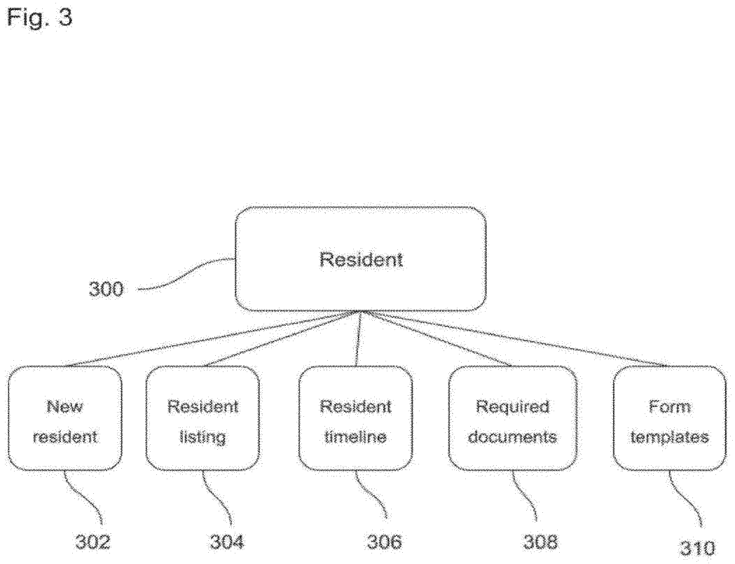

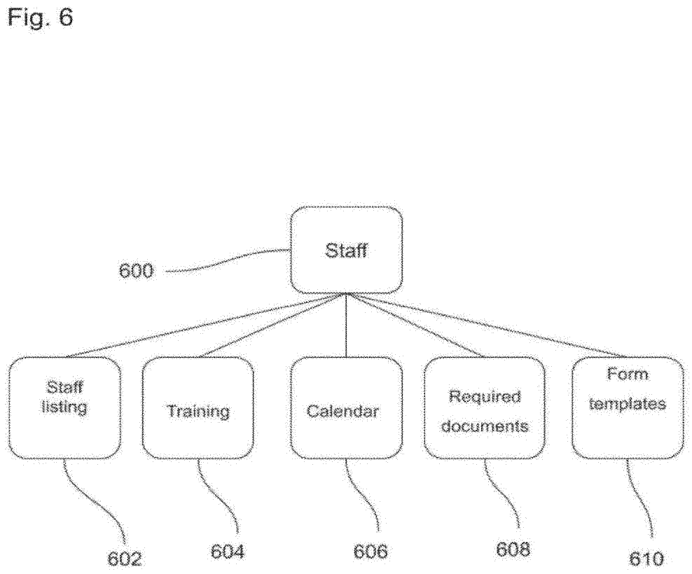

Referring to FIG. 3, the resident module 300 allows the care home to enter and maintain records for each of their residents 300 in a central location. This information is then available throughout the rest of the system and is also conveniently located for the staff 600 as shown in FIG. 6 to find in the event of an emergency.

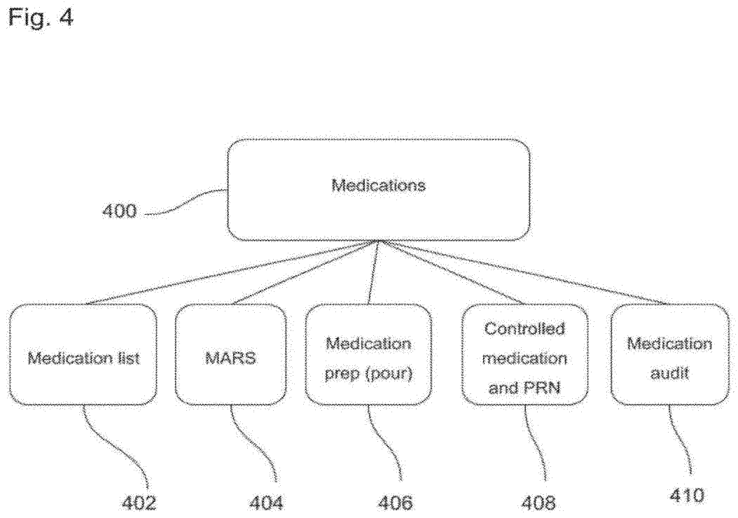

This module also provides medical personnel to enter relevant information regarding the resident's care into the system that will then be fed into the other relevant modules of the website like the medication module 400 as shown in FIG. 4. As residents 300 move in and move out, electronic records will be maintained for audit purposes.

The resident module 300 contains five sections, new resident 302, resident listing 304, resident timeline 306, required documents 308 and form templates 310 as following: New resident new resident information, this section contains four tabs, Identification, and info (LIC 601), Appraisal (LIC 603a), Physician report, and accommodation and fees.

### Resident Listing

The resident listing section 304 lists any resident after they have been entered in the new resident section, in this section a user can click the residents name and it will takes him back to the above page so he can alter resident information, the resident listing section 304 also shows a birth date, sex, and the admission date.

### Resident Timeline

The resident time-line section 306 is a feed of any caregiver portal entry ADLs 502, medication 506, admin 800, care note 504 and treatment.

### Required Documents

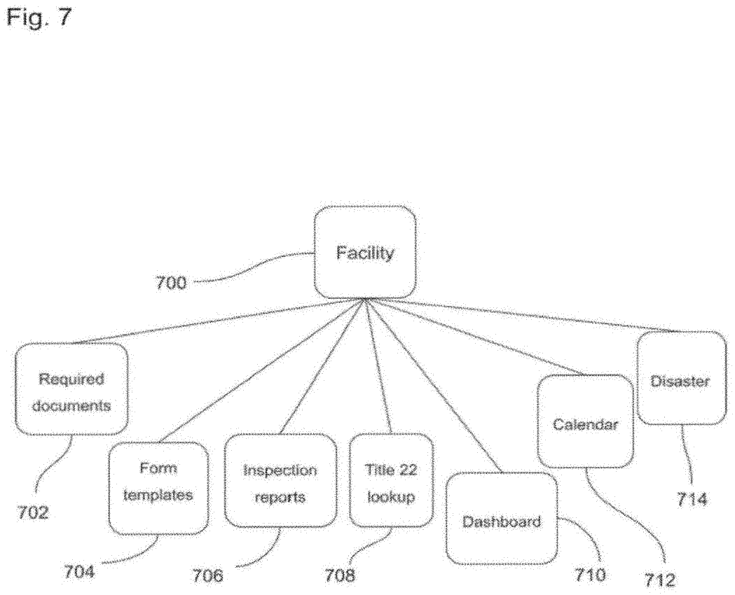

The required documents section 308 is where a user uploads and view required documents listed on California state form LIC 311 for each facility 700 as shown in FIG. 7. Here the user will upload each required document 308 for each resident 300, it can be viewed or upload. As upload user will enter an expiration date as well as a signature date, when completed a green check mark will show under the word present to show compliance. This is an easy to view screen all residence and what is present and what is missing. This page contains resident name, whether the item is required, whether it is present, and what action to take including view upload and print. The documents and templates in the sections of required documents 308 and form templates 310 are:

1. LIC 603 Resident Appraisal ARF Resident

2. LIC 603A Appraisal RCFE

3. LIC 625 Appraisal Need and Services Plan

4. LIC 602A Physician's Report for RCFE Physician's

5. LIC 602 Report for ARF

6. LIC 601 Identification and Emergency Information

7. LIC 604A Admission Agreement

8. LIC 613C Personal Rights Information

9. LIC 405 Residents Safeguarded Cash Resources

10. LIC 621 Resident Personal Property and Valuables

11. LIC 624 Unusual Incident/Injury Report

12. LIC 624A Death Report

13. LIC 622 Centrally Stored Medication and Destruction Record Resident

14. LIC 9060 Theft and Loss Record

### Medication Module

All of the medications 400 in the facility 700 as shown in FIG. 7 are to be entered in the Medications module 400. Each medication 400 is associated with the resident 300 who will be taking the medication 400 as well all the relevant information (dosage quantity and frequency, who prepared/poured the medications 400, who passed the medications 400 to the resident 300, etc.). The medication module 400 also allows tracking the medications 400 if the resident 300 leaves the facility 700 or if the medication 400 is destroyed (in the event of death).

There is in depth reporting to indicate when medication 400 was prepared but never passed to the resident 300, there is alerts built in if the resident's medication 400 is nearly empty and refill needs to be ordered. Built in reporting allows the care home administrator 200 as shown in FIG. 2 to monitor the controlled medications 400 to ensure that they are not being mishandled or stolen. The system also restricts the care home staff 600 from pouring too much medication 400 or passing the medication 400 too frequently. There are also reminders built in if a dose was missed or if the resident 300 refused to accept the medication 400.

The medication module 400 contains five sections, medication list 402, MARS 404, medication prep (pour) 406, controlled medication and PRN 408, and medication audit 410 as follows:

### Medication List

In the medication list section 402, a user enters a new drug and the required information, then it is automatically transferred to the mars 404 and stored meds LIC622 form. Here user can see an entire list of medications 400 in the facility 700 or break it down per resident 300 this is also where a user add medication 400 when clicking add medication screen comes up and collects all data on every prescription. This includes resident name, prescription type, medication type, dosage frequency, dosage strength, quantity, unit, supply quantity, instructions, expiration date, date filled, date started, and date, prescribing physician, pharmacy name, prescription number, number of refills, and refill expiration you can also check the box controlled medication or PRN medication 408.

### MARS

In the MARS section 404, MARS stands for medication administration record sheet, there are thirty one boxes that go across and there is a spot for each medication 400 to be listed each thirty one box represents a date the medication 400 was given the signatures will go from the caregiver med pass to the MARS sheet eat 404. On the screen the user will choose the resident 300 and the month these MARS 404 available for reprint anytime.

### Medication Prep (Pour)

In the section of medication prep (pour) 406 (Prep means to prepare medications 400. Pour is to pour from a bottle or remove a tablet from a bottle. Some facilities say to “prepare medications” and some say “Pour medications” they are both the same essentially). User reads screen and sets up doses of medications 400, checks that they are set up. This information goes to caregiver portal medication 506 pass.

### Controlled Medications and PRN

In the section of controlled medications and PRN 408, when a user clicks here, he will be brought to either his controlled medication sheet or PRN sheet with the resident 300 drop-down menu on top.

### Medication Audit

The Medication audit section 410 will contain any medication errors missed dosage per resident 300.

### Caregiver Portal Module

The caregiver's module 500 provides a single focused tool for caregivers 500 to perform their duties and to have everything documented in the system. This not only provides a record of the activities for future reference, but it also provides reminders and alerts when appropriate (i.e. a reminder to check on a resident in two hours after giving them pain medication 506 to see if the medication 506 was effective).

This module is designed to be a one-stop-shop for caregivers 500. These screens give them access to track AOL's 502, provide care notes 504 throughout the day, pass medications 506 to the residents 300 (as shown in FIG. 3), and it also provides a time-line 508 which summarizes the residents' care over time. The timeline 508 also provides a way for a care homeowner or administrator to monitor the overall wellbeing of the care home's residents.

In the Caregiver 500 module, a user has four sections, AOL's 502, care notes 504, medication 506, timeline 508 and resident profile 510 as follows.

### AOLs

The AOL's section 502 will have a full list of all AOLs 502 including self-administration of meds, treatments, eating, escort to meals, snacks, room tray, dressing, clothing choice, change depends/diapers, perry wash, sponge bath, full shower, oral care, hair care, transferring, toileting assistance, urine, bowel movement, recreational activities, escort activity, appointments, change sheets, housekeeping, laundry, tidy room, dentures, hearing aid karma glasses, wait, blood pressure, blood sugar, INR Coumadin level, outside care or visitor, passive supervision, safety check, walking, social services, transportation. The next column has all drop down menus to select independent, refused or staff assist. The next row is an area to write notes and above that all is where a user selects his resident.

### Care Notes

In the care notes section 504 there is a select resident button care note upload file and a check-box for submit administrator review if this box is checked in immediately goes to the administrator's dashboard 200 with an alert 202 as shown in FIG. 2.

### Medication

In the medication section 506, a user will click the resident 300 and it will automatically generate the medications that are beat to be passed at what our what the name of them are and have a check-box for the caregiver 500 to check past or refused there is a note section and the unique dispenser code.

### Timeline

The time-line section 508 summarizes the residents' care over time. The timeline 508 also provides a way for a care homeowner or administrator to monitor the overall wellbeing of the care home's residents.

### Resident Profile

The resident profile section 510 collects data from intake or new resident section 302 as shown in FIG. 3 photo and several other places to create easy to use resident profile sheets for staff.

### Staff Module

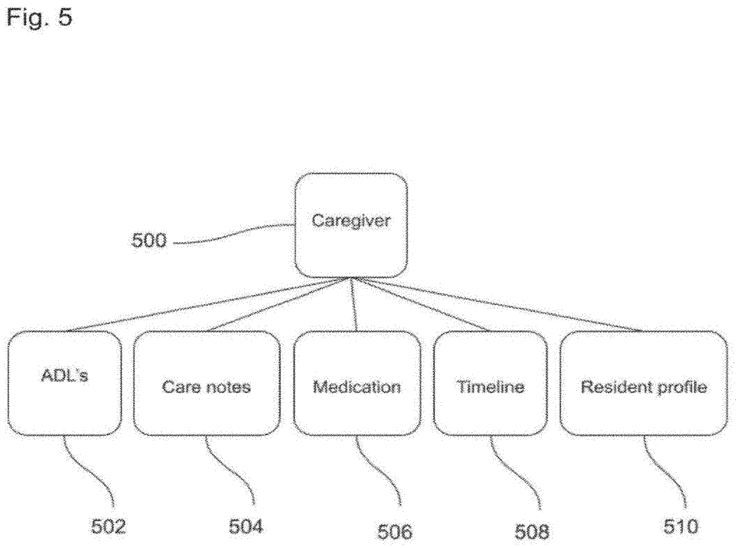

The staff module 600 provides a central location for entering and referencing the staff contact information, training documentation, and provides scheduling and basic HR information. Each of the care home staff members 600 are setup with a profile and are granted specific access by their administrator. If they are a caregiver 500 as shown in FIG. 5, they will just have access to the caregiver module 500 as shown in FIG. 5 which allows them access to perform their day-to-day functions but does not allow them access to other staff members data or private resident data (i.e. SSN). A staff member 600 setup as an administrator will have full access to all the application at the care home level.

The staff module 600 also provides a place to track all the staff members' training 604 to ensure compliance with the training requirements. The staff module 600 contains five sections, staff listing 602, training 604, calendar 606, required documents 608, and form templates 610 as follows.

### Staff Listing

In the staff listing 602 there is a button that says add staff, when the user press add staff, the user will see a List of all staff members.

### Training

In the training section 604, the user clicks add training and fill out a training log that includes staff member, training types, training title, training description, completion date, renewal date, it also has an upload button.

### Calendar

In the calendar section 606, the user will find a calendar for activities, meds, doctor appointments, safety checks, and night changes meetings.

### Required Documents

The required documents section 608 contains the staff required documents 608 they are the same as resident required documents 308 as shown in FIG. 3.

### Staff Required Documents

1. Health Screening Report/Tuberculosis (TB) Clearance-Facility Staff

2. Tuberculosis (TB) Clearance and “Good Health” Statement-Volunteers

3. Reports of Actual Hours Worked by Staff

4. LIC 501 Personnel Record

5. Age Verification/Copy of Current ID

6. Administrator Certificate

7. Documentation of Annual 20 clock-hour Training

8. LIC 508 Criminal Record Statement

9. Criminal Record Clearance or Exemption

10. Verification of Staff Training

11. Verification of CPR Training

12. Verification of First Aid Training

13. Staff Credentials/Certifications

### Form Templates

In the form templates section 610, there is the LIC501 form, the user will fill out the staffs first name, last name, birth date, phone number, email address, address line, city, zip, you will assign a user ID, a sign password, confirm password, last physical exam, last TB test, date of hire, signature date, and the user will have the option to choose the form lic501.

### Facility Module

The facility module 700 allows user to setup and maintain all the information regarding their facility 700 in the system as well as upload all the required documentation into one location. The facilities information is used for automatically determining compliance based on the classification, number of beds, type of patients (i.e. dementia, bedridden, etc.). The facility module 700 contains 7 sections, required documents 702, form templates 704, inspection reports 706, title 22 lookup 708, dashboard 710, Calendar 712 and disaster 714 as follows.

The required documents section 702 include:

1. RCFE License

2. LIC 500 Personnel Report

3. Verification of First Aid Training for Staff Providing Care

4. Written First Aid Procedures

5. Menus (16+ Residents: Min 30 Days History; <16 Residents: Sample Menu)

6. LIC 61OE Emergency Disaster Plan

7. LIC 401 Financial Records Including Income and Expenditures

8. LIC 402 Surety Bond

9. Plan of Operation: Plan for Staff Trainings

10. Plan of Operation: Facility Sketches

11. Plan of Operation: Statement RE: Handling Client Monies

12. Plan of Operation: Plan for Hazardous Behaviors

13. Plan of Operation: Policy for Facility Visits

14. Neighborhood Complaint Policy

15. Subscription to Service for Changes in Requirements

16. Insurance Statement

17. Fire Inspection Report

18. 16+ Residents: Dated Weekly Employee Time Schedule

19. Licensee Affidavit on Persons Exempt from Fingerprint Requirements

20. Appropriate Driver's License for Each Staff Member Transporting Residents

21. 7+ Residents: Notices of Planned Activities (Min 6 Months History)

22. 50+ Residents: Program of Activities

23. 50+ Residents: Evidence of Consultation from Nutritionist, Dietitian, or Home Economist

24. LIC 610 Emergency Disaster Plan

25. LIC 215 Applicant Information

26. LIC 200 Application for Community Care Facility

27. LIC 9092 Fire Pre-Inspection/Consultation Request

28. LIC 309 Administrative Organization

29. LIC 198 Child Abuse Central Index

30. LIC 400 Affidavit Regarding Resident Cash Resources

31. LIC 308 Designation of Facility Responsibility

32. LIC 9054 Local Fire Inspection Authority Information

33. LIC 9020 Register Of Facility Clients/Residents

34. LIC 401a Supplemental Financial Information

35. LIC 403 Balance Sheet

36. LIC 404 Financial Information Release And Verification

37. LIC 999 Facility Sketch (Floor Plan)

38. Board of Directors Statement (if applicable)

39. Corporation Bylaws (if applicable)

40. Dementia Plan of Operation

41. Hospice Waiver Documentation

42. Theft and Loss Policy

43. Admission Agreement

The form templates section 704 includes blank forms for use. In the inspection report section 706 a user uploads inspection reports and dates when inspection was performed. In the title 22 lookup section 708 (Title 22 Regulations of the California Code contains rules concerning community care facilities in the state.) Child Care and residential care facilities fall under the purview of the Community Care Licensing Division and must follow these regulations. Facilities for children, the chronically ill and the elderly, along with foster homes and social rehabilitation facilities, title 22 for licensing and operating regulations. Operating rules for each type of facility 700 include staffing requirements, staff-resident ratios, and resident rights.). PDF of title 22 with the option to click the find box and type in subjects for title 22 regulation numbers.

### Dashboard

In the dashboard section 710, there are administrator alerts, care notes feed under administrator alerts, all alerts regarding missing documents, medications becoming due, incident reports, anniversary dates, and birthdays.

### Calendar

In the calendar section 712, the user will find a calendar for activities, meds, doctor appointments, safety checks, night changes, and meetings. Calendars are for staff, residents, and facility.

In the disaster section 714, comprehensive disaster planning and all information required for AB 3098 RCFE (AB 3098 RCFE stands for Assembly Bill No. 3098. residential care facilities for the elderly, emergency, and disaster plans.) disaster plan requirements including resident forms lIC 603 and lIC 622, facility forms lIC 9020 and lIC 500.

The disaster required documents are:

1. LIC 500 Personnel Report

2. LIC 61OE Emergency Disaster Plan

3. Plan of Operation Facility Sketches

4. Fire Inspection Report

5. LIC 603A Resident Appraisal RCFE

6. LIC 625 Appraisal Need and Services Plan Identification and Emergency Information

7. LIC 601 Centrally Stored Medication and Destruction Record Resident

8. LIC 622 Theft and Loss Record

9. LIC 9060

### Admin Module

The admin module 800 contains two sections, users 802, and system settings 804 as follows.

### Users

In the user section 802, a user will create user IDs for facility 700 (as shown in FIG. 7). System settings The system settings section 804 is to collect all data and converts into a report.

### Licensed Program Analyst (LPA) Module

The Licensed Program Analyst (LPA) module 900 provides a concise view of the facilities required documents allowing an LPA to quickly identify areas of the facilities documents that is potentially out of compliance and should be reviewed. The system and methods for ensuring facility compliance plans to build a compliance dashboard which will summarize several of the key metrics and activities of the facility inspection process to audit for compliance and/or for insurance companies to determine the overall risk and compliance of a facility.

This compliance dashboard will allow the state to view all facilities. With this functionality, the state can prioritize which care homes to audit earlier based on overall risk. It would also be possible to assign LPA users to specific care homes and/or counties within the state that they are responsible for reviewing. In the licensed program analyst (LPA) module 900 there is a username and password given to LPA who can then log in and view only required documents section of the program. The LPA will have access to all facility 700 as shown in FIG. 7, Staff 600 as shown in FIG. 6 and resident required documents 308 as shown in FIG. 3.

### Emergency Patient Transfer System

According to at least one embodiment of the invention described in enabling detail herein, the inventor provides a unique network-based system for requesting emergency transfer of residents of care homes and other residential care facilities from original or current facilities to available facilities.

It is a goal of the present invention to enable a resident of a home care facility to be transferred from a home facility to an available facility in a manner that is computer aided reducing task performance work for operators seeking to request and initiate transfers. It is another goal of the present invention to enable operators to initiate resident transfers from home facilities or current facilities to other facilities from a remote location not connected physically to the home or destination facilities. A further goal of the present invention is to provide updated information on facility capabilities and bed, room or unit availability information to operators seeking to request transfer of a resident as available data that may be accessed remotely by an operator from a visual Geo-representation of an area containing the facilities. The present invention is described using the following examples, which may describe more than one relevant embodiment falling within the scope of the invention.

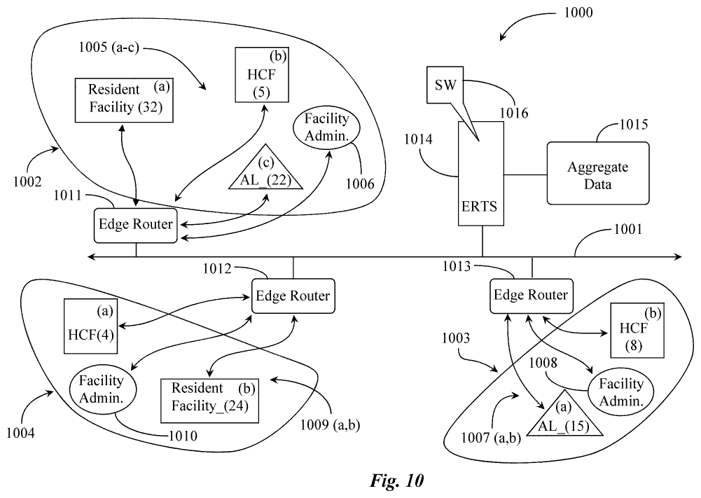

FIG. 10 is an architectural overview of a communications network 1000 supporting computer aided transfers of residential care facilities or homes according to an embodiment of the present invention. Network 1000 is characterized in this architecture by a network backbone 1001 representing a wide area network (WAN) like the Internet Network. Network backbone 1001 represents all the lines, equipment, and network-access points that make up the Internet Network including connected sub-networks. There are no geographic limitations to the practice of the present invention though a preferred embodiment may include all registered facilities in a State. Other embodiments may have networks overlapping State boundaries like in Tri-State regions where locality is considered geographically feasible for a residential transfer.

Network backbone 1001 supports edge data routers 1011, 1012, and 1013 in this embodiment; the data routers are equipped to provide sub-networks like a sub-network 1002, a sub-network 1003, and a sub-network 1004 connection to and access to the overall network. Sub-networks 1002, 1003, and 1004 may be mobile telephone carrier networks, local area networks (LAN) with access to the Internet Network, wide area networks (WAN) with access to the Internet Network, or a combination of those.

In sub-network 1002, care facilities 1005 (a-c) are depicted connected to network backbone 1001 via Internet capable access equipment and an internet service provider (ISP) through edge router 1011. Facilities 1005 (a-c) may represent varied types of facilities that care for residents without departing from the spirit and scope of the present invention. For example, facility 1005 (a) may be a residential facility having capacity for thirty-two live-in residents, and facility 1005 (b) may be a home care facility (HCF) which is much smaller than a multiple unit residential facility with capacity for five live-in residents. Facility 1005 (c) may be an assisted living facility (ALF) where residents have units with privacy like in an apartment complex. In this case, assisted living facility 1005 (c) has twenty-two units for live-in residents.

A facility administrator (Facility Admin.) 1006 may be anyone authorized to request and initiate the transfer process of a resident from one facility to another facility. Administrator 1006 may access network backbone 1001 from any location using a connected mobile communications device capable of accessing a data network like the Internet Network using an internet service provider and a local network browser application.

Sub-network 1004 includes facilities 1009 (a,b). A home-care facility 1009 (a) is depicted as having capacity for four live-in residents. A residential facility 1009 (b) is depicted having a twenty-four resident capacity. A facility administrator 1010 is depicted and may be anyone who is authorized to request and initiate the transfer process of a resident from one facility to another facility as mentioned above with respect to administrator 1006.

Sub network 1003 includes facilities 1007 (a,b). AL facility 1007 (a) may be an assisted living community with a capacity to house fifteen residents. Facility 1007 (b) may be a home care facility having a resident capacity of eight residents. A facility administrator 1008 may be an administrator or another authorized individual working from home using a mobile device connected to the Internet Network without departing from the spirit and scope of the present invention.

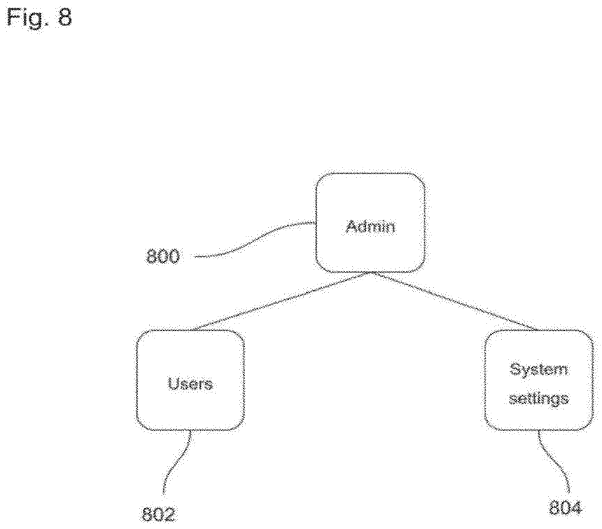

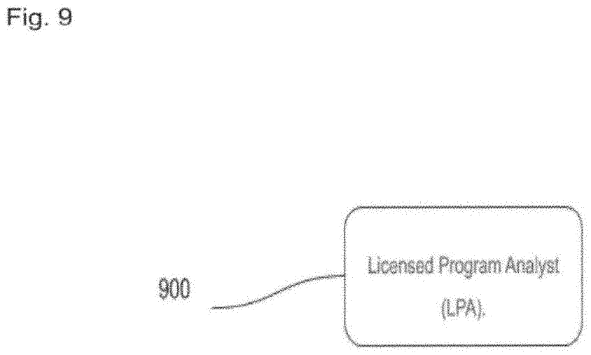

In this example, all of the visible facilities may be connected to a shared network portal server referenced herein by an emergency resident transfer server (ERTS) 1014 where each facility may manually or automatically upload by programed sequences of all of each facility's current and pertinent information such as current resident identification, resident care profiles, resident medications and treatments, facility licensing and compliance information, facility property data, facility emergency data, and other necessary information on a regular basis as described further above with respect to a portal system for ensuring facility compliance with State and Federal standards covered by the description of FIGS. 1-9.

All the facilities depicted in coverage areas of sub-networks 1002, 1003, and 1004 may be represented from a Geo-perspective by including them on an accessible representation of geography like a digital map of a state or region containing the facilities. In one embodiment, authorized agents working for a facility from any of the mapped facilities or from an unmapped remote location may access a Geo-representation (typically a digital map) of an area containing facilities, and may browse those facilities by interacting with interactive icons overlaid on the Geo-representations where those icons are associated with meta-data about that facility. Icons representing facilities may vary in visual depiction of the facility to reference facility type or category; for example, an apartment complex, a home, and a commercial building that houses residents may have separate icons for visual sorting purposes.

Network backbone 1001 supports emergency resident transfer server (ERTS) 1114 hosting a software (SW) application 1016. Server 1014 functions as a portal accessible through all of the residential facilities 1005 (a-c), 1007 (a,b), and 1009 (a,b) depicted as well as for all the remote facility administrators 1006, 1008, and 1009 working from other remote locations that are not defined as within one of the facilities.

Server 1014 has connection to at least one Aggregate Data 1015 adapted to hold the current and latest aggregate data from all the facilities. The aggregate data may include, but is not limited to, facility type, category type, resident capacity, current number of residents present, resident identification, staff identification, transfer service utilities and data, medical profile for each resident, equipment or service requirements data for each resident, medications required for each resident, current mobility ratings for each resident, physical residential property information, medical and equipment capacity of each facility, state license and compliance status information for each facility, generic facility data like facility address, contact information, resident vacancy, and medical professionals available for on-site evaluation and treatment. SW 1016 running on server 1014 may return search results to authorized users operating from any one of the facilities or from a remote location. SW 1016 may provide data synchronization services for facilities to acquire the most current information and changes made to all facets of the facility and the residents thereof. Server 1014 aided by SW 1016 may be accessed using typical browser-based software from a network-connected computing device or mobile device having internet browsing capability.

In one embodiment, server 1014, aided by SW 1016, is a function of a network cloud-based service storing and synchronizing data centrally for all facilities registered within the service authorized to access the server or authorized to access the server through an authorized agent that may be accessing remotely from an off-site facility. SW 1016 may include an information page-generation capability to serve each authorized administrator a personalized dashboard displayable through browser-based SW using an internet capable device with input and display functions.

SW 1016 may include a semi-automated process for receiving requests for transfer of residents, for qualifying the practicality of requests for transfer, and for initiating the required processes necessary to complete transfers including scheduling, selecting and initiating transportation modes, transferring required forms and records, and updating facility status after transfers have been confirmed and initiated.

In the general use of the present invention, an authorized administrator or agent working for any of the facilities may log onto portal server 1014 aided by SW 1016 and make a request to transfer one or more residents from a home facility that may be under an evacuation order due to an emergency, for example, multiple transfers may be transferred to multiple destinations. An emergency for which a transfer may be requested might be fire, storm, earthquake, power outage, contagion, or chemical emergency.

Server 1014, aided by SW 1016, has access to the most current data of all the potential destination facilities including the source facility. SW 1016 may perform matching of each resident's medical needs and conditions to the capabilities of potential destination facilities that may receive, qualify, and confirm the transfers. The fifteen residents of assisted living facility 1007 (a) may all be transferred together to facility 1005 (a) if fifteen of the thirty-two units, rooms, or beds happen to be listed as unoccupied, appropriate, and available to fill.

An operator may hand-pick facilities on a displayed map using a visual browsing technique to identify facilities on the map by icon (color or no color) and the initial generic meta-data that may be displayed by right-clicking, double-clicking, or hovering a pointer on an icon where those data show one or more vacant units, rooms, beds, and spaces available. In one embodiment, the operator requesting transfer may browse meta-data on any facility by clicking on appropriate links displayed in the operator's browser window.

In one embodiment, the operator may elect to have SW 1016 execute a data search of Aggregate Data 1015 about potential destination facilities based on matching resident data where the resident data includes critical information about care, medications, mobility, oxygen requirements, dialysis requirements, doctor visitation requirements, etc. In such an embodiment, a rejection of the request, or an acceptance of the request, may be a result of analysis performed by SW 1016. In the case of denial of a request, Aggregate Data 1015, aided by SW, 1016 may provide one or more reasons why a requested resident transfer is denied where one or more of those reasons result from data comparison between the two facilities. In other embodiments, an operator may access the same data and perform manual research such as looking at a facility map and the qualifying presence of required equipment for a resident with special needs.

In one embodiment, residents requiring a transfer due to an emergency may also be distributed to temporary facilities like shelters or facilities that do not keep residents more than a select period of time and later transferred to a more appropriate or permanent facility.

In some emergency situations, some, but not all, residents of a facility may have to be transferred to other facilities if, for example, they become infected with a virus and the facility wishes to limit spread to other residents in a same facility. SW 1016 may create, update, or change existing data with or without human intervention so that ball administrators or authorized personnel requesting, transporting, and receiving transfers may have access to the latest and most current data.

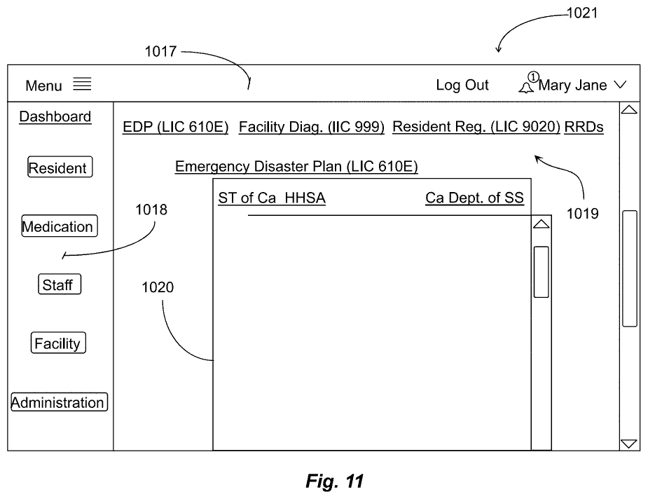

FIG. 11 is a screen shot of an emergency resident transfer (ERT) dashboard panel 1021, similar to the dashboard panel of FIG. 1, created by SW 1016, is depicting an emergency disaster plan (EDP). Screen shot of dashboard panel 1021 represents a user interactive interface (UI) dashboard panel that may only be accessed by authorized individuals approved for accessing certain resident and facility information and for determining correct and safe metrics for transferring residents from one facility to another facility using the system. Dashboard panel 1021 includes a title bar 1017 containing at least an interactive menu, a log-in/log-out utility, a notifications list icon, and a level of personalization once the operator has been validated to operate through the site. Validation might include password and personal identification number (PIN), and other secure identification or validation methods known in the art and available to the inventor.

The menu icon on the title bar 1017 may list several options available to an authorized administrator. In this view, the operator has interactive options 1019 arrayed across the top of the display just under title bar 1017 with an emergency disaster plan, EDP (LIC 610E), open in a scrollable position on the operator's screen. The operator may also select from other display options: facility diagram, Facility Diag. (LIC 999), that may show a detailed view of the current floor plan, equipment, and medical capabilities of a facility. Other metrics may include the total number of residents, Registered Reg. (LIC 999), emergency disaster plans, and any vacancies in residency identified as available rooms. Interactive options 1019 may also include a link to a list of resident-required documents (RRDs) that may be needed for transfer, treatment, power of attorney, etc.

Dashboard panel 1021 includes sidebar 1018 in this example to display interactive links to other parts of the system where information is available. A resident option (top) allows the operator to call up and display a current resident register identifying all the current residents of a facility being reviewed or looked at for a potential destination for one or more transfers. A medication option (next down) enables an operator to see the medications and treatments a resident is prescribed and to determine if such medications are on order, in review, in resident or nurse custody, or in re-evaluation.

Sidebar 1018 may also include a staff option that the operator may click to see all the working staff at a facility including qualifications, shift hours, etc. An interactive link labeled facility may also be provided within sidebar 1018 and may be a link for the operator to pull up any floor plans for residents in a same region. Sidebar 1018 may also host a facility option; the facility option may be invoked to see further details about a single facility such as a facility floor plan, facility ratings and revues, facility medical capabilities, facility emergency plan, etc.

Sidebar 1018 may also host an administration option enabling the operator to call up meta-data about an administrator of a facility or a list of facility administrators if there is more than one to a facility. Such meta-data may include hours, contact information, credentials, emergency contact data, and the like. In one embodiment, administrators may contact, associate, and communicate with one another via system messaging, chat, video chat, telephone, etc. In this view, an emergency disaster plan (EDP) 1020 of a facility is open in screen and is scrollable through all parts of the plan.

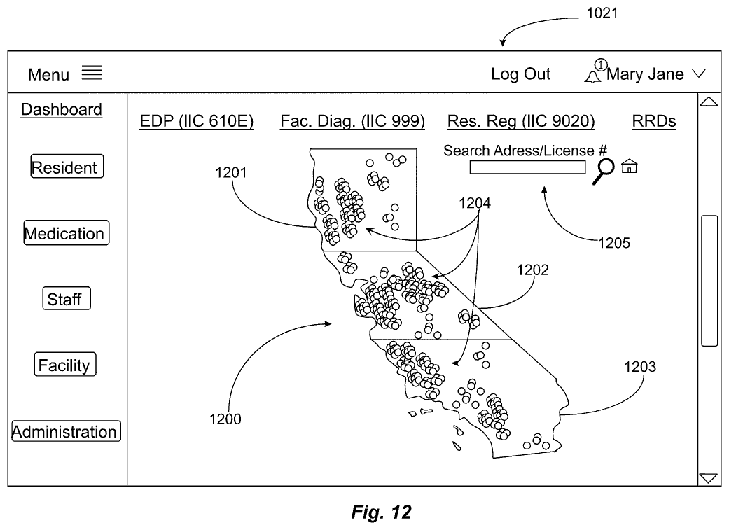

FIG. 12 is a screen shot of an ERT dashboard panel 1021 of SW 1016 of FIG. 1 depicting a state map of facilities registered on the network. In this view dashboard panel of SW 1016, an option to select and display a digital map 1200 is provided. Digital map 1200 is of the state of California in this embodiment but may be any other state having facilities that are registered on the network. Digital map 1200 may be an interactive map with interactive data that an operator may select to view more information. Digital map 1200 may be provided by a mapping service that provides mapping services to the system of the invention including allowing the maps to be made interactive to enable navigation to additional meta-data.

In this embodiment, digital map 1200 of California is sectioned into three navigable areas. These are Southern California 1203, Central California 1202, and Northern California 1201. An operator in Southern California who needs to transfer residents because of a disaster, or some other emergency may determine to select a facility in the same area but outside of the emergency or disaster zone for example. Overlaid on digital map 1200 are, in this embodiment, interactive icons 1204 representing all the facilities that are available as participating facilities in the system. Facility icons may vary in shape, size, and or color, to represent different types of facilities included in the overlay layer. Icons 1204 may overwhelmingly represent residential care facilities in number, but may also include emergency response facilities, fire stations, sheriff's offices, police departments, hospitals, and temporary shelters. Each icon on digital map 1200 may be zoomed in on and manipulated to reveal data about that facility. Each icon is associated to digital map 1200 by location and first level meta-data may include facility address, facility name, number of residents, status relative to vacancies, etc. An operator may select any facility on map 1200 and obtain all pertinent and current data about that facility in deciding whether the facility may be a potential destination facility for one or more residents the operator may need to transfer. In one embodiment, the operator may be an administrator or agent working for a facility having one or more residents that need to be transferred.

Digital map 1200 may be displayed with overlaid data (icons) that are included by default or ordered by the operator because of a search activity. In this embodiment, dashboard panel 1021 includes a search utility 1205 that enables an operator to call up the facility icons that match a certain search criterion. For example, facilities may be searched through digital map 1200 as an interface where the overlay layer is created on the fly because of matching facilities to criteria entered in a search utility 1205 by the operator. An operator may, for example search facilities based on facility address, facility name, facility size, facility vacancy status, facility proximity to emergency response facilities or hospital or ambulance services, facilities having specific equipment and licensed staff, and so on.

In one embodiment, an operator may select a facility to enable all the interactive options in dashboard panel 1021 to be associated with that selected facility. In this way, the digital map interface is a user interface for navigating required information to determine if a facility meets the requirements of one or more residents that the operator needs to transfer. In a preferred embodiment, the system is set up for emergency transfer or residents because of a necessity of the one or more residents to be transferred out of a facility or facilities collectively. However, that should not be construed as a limitation of the invention as it may also be used to place residents that may be new residents looking for a first facility or residents who have outgrown a previous facility because of a new need for equipment, expertise, or services, the current facility did not provide.

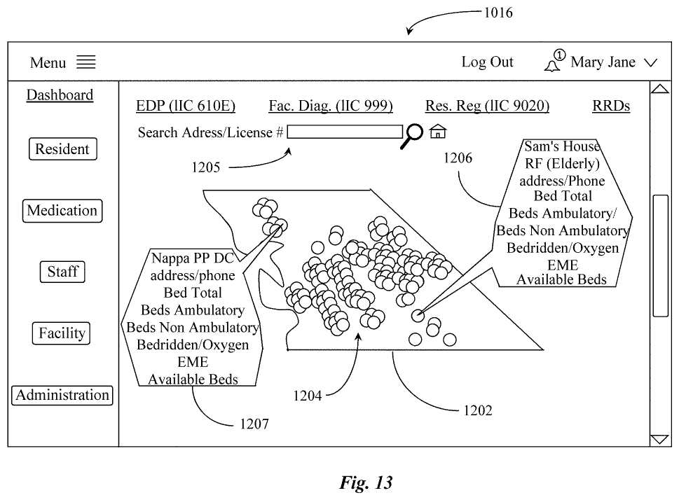

FIG. 13 is a screen shot of an ERT dashboard panel of SW 1016 of FIG. 1 depicting a selected region of the state map of FIG. 12. In this view, an operator has selected a region 1202 of digital map 1200 of FIG. 12. Map region 1202 represents the region of Central California in this example. An operator may, in one embodiment, first use search utility 1205 to cull the list of icons 1204 that are displayed in the digital overlay of the map. One with skill in the art of digital map services will appreciate that options for zooming, street views, satellite views, etc. are typically available to the navigator of the map or region of the map. For example, an administrator transferring a resident may command a street view of the facility and see the exact location and what it looks like from a perspective of one or more fronts on the ground.

In this embodiment, a facility selected results in a pop-up visual information box 1206 containing frost layer meta data about that facility like the type of facility and the name of the facility. The name of the facility is Sam's House. Information box 1206 may include the address and telephone and contact data for the facility and the total number of beds the facility has capacity for. Of the total number of beds, the information box 1206 may further reveal which of the total beds are ambulatory and non-ambulatory.

Information box 1206 may also reveal the number of residents who are bedridden and need oxygen or other treatments while bed ridden. Information box 1206 may reveal if there is emergency medical equipment (EME) on site at the facility or not. Information box 1206 may also be programmed to reveal the latest updated residency state at the facility, for example, revealing several beds that may be vacant and could accept a resident being transferred.

Information box 1207 represents the same overly of updated meta data as does box 1206 but for another facility selected. In a preferred embodiment, the act of selecting an icon 1204 from map region 1202 enables the operator to utilize SW 1016 as a user interface linking the available interactive options described previously to data relevant to the selected facility that is stored in a data repository and frequently updated by an authorized agent or automated process of that selected facility.

In actual practice, the operator will select one facility from the map and then use the dashboard panel to navigate the more granular data about the facility including licensing data, forms required, facility floor plan, facility administrators, facility staff and schedule, residents living there, medications and care charts, reviews and audit information, and more granular data as may be deemed appropriate to make accessible to an authorized agent or administrator working to care for residents.

In actual implementation, a doctor may be authorized to transfer residents who have medical needs whereas a facility administrator who does not have license may be authorized only to transfer residents with no adverse medical requirements. In one implementation it may require more than one person to sign off on a new facility selected to receive one or more transferred residents. Certain private information about residents may be encrypted and not made available to agents or administrators who are transferring residents.

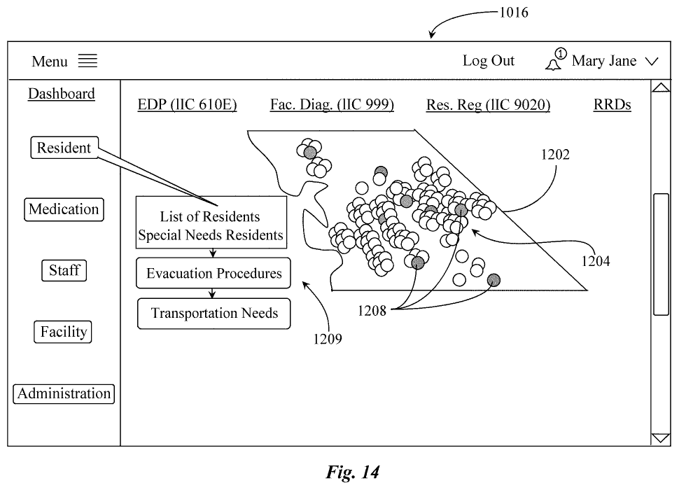

FIG. 14 is a screen shot of an ERT dashboard panel of SW 1016 of FIG. 1 depicting the selected region of FIG. 13 highlighting available destination facilities. In one embodiment, icons 1204 in map region 1202 may be customized by a search function or the like to display icons 1208 differently such as by another color indicating residential facilities that have current vacancies for example. Other metrics might be considered in such a customized return such as facilities having vacancies and necessary medical equipment on site.

In one embodiment, a residential facility may publish an upcoming vacancy or vacancies where the color of the icon is different than a facility with immediate vacancies. There are many variations. Icons may be ordered of differing shapes, differing colors, and differing sizes. Icons of different colors and or shapes may be assigned to different types of residential facilities. Differentiation may be ordered for sheriff stations, fire stations, hospitals, etc.

In one embodiment, when a facility is not linked to on the network by way of selecting its icon through the dashboard panel (SW 1016), then the agent or operator may navigate his or her own facilities by default using the various interactive options provided in the interface. For example, information boxes 1209 may provide additional data from interaction with option “resident” in the panel side bar like a full list of residents needing transfer, the portion who have special needs, the evacuation procedures that are to be followed, and details about the transportation needs or requirements to get the residents into the new facilities. In one embodiment, the process for transferring one or more residents from one facility to another is a semi-automatic process to a degree or level that may be selected by the operator through the dashboard panel.

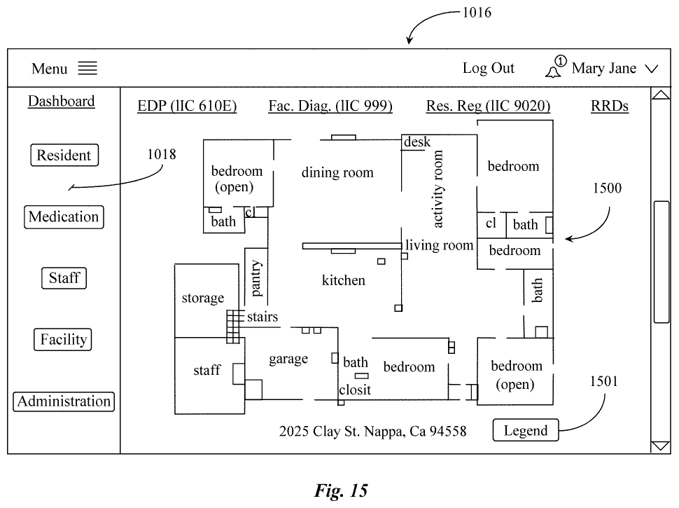

FIG. 15 is a screen shot of an ERT dashboard panel of SW 1016 of FIG. 1 depicting a facility diagram. In this embodiment, an operator has elected to view a facility diagram of a facility he or she is evaluating as a potential destination facility to receive one or more transferred residents. All registered facilities may have one or more floor plans available to authorized individuals working to care for and transfer residents. Floor plan 1500 may be ordered by interaction with the option facility anchored within side bar 1018, or by selecting Facility Diagram at the top of the panel. Facility diagram 1500 is in this example an exemplary floor plan of a facility with the address 2025 Clay St. Napa, Calif. 94558, a facility selected from the central region 1202 of 1200 described further above in FIG. 14.

Floor plan 1500 may be a single level floor plan or a multilevel plan where one level may be selected and displayed at a time. In one embodiment, multiple levels may be displayed in windows overlaying one another. Floor plan 1500 may include icons and symbols that may be defined in a legend 1501 aiding the operator in identifying important aspects of the facility as evidenced in the floor plan. In this plan there are five bedrooms on a single ground level, two of the five bedrooms identified as open or vacant.

The administrator working on a transfer may interact with floor plan 1500 much in the same ways described above with the state map 1200 introduced in FIG. 12. For example, the administrator may see space dimensions for rooms, bathroom and storage arrangements, activity room dimensions, kitchen facility, and other features. The administrator may also select icons to view capacities and capabilities like medical equipment on site, medical staff, medical pharmaceutical storage, safety equipment, access ramps, normal and emergency exists, emergency supplies, generators, water heaters and other features.

In one embodiment where outside features are available on the land occupied by a facility, outside feature additions (plans) to a basic floor plan may also be provided with the floor plan or set of floor plans and may include propane tank identification and location, outside storage, outside electrical and sewage hook ups for mobile units, outside activity areas like spa areas, or pool areas, and other like features. Plan overlays may be provided that contain interactive links to additional meta data about a feature visible or represented by an icon defined in legend 1501 selected by an operator browsing a floor plan like plan 1500.

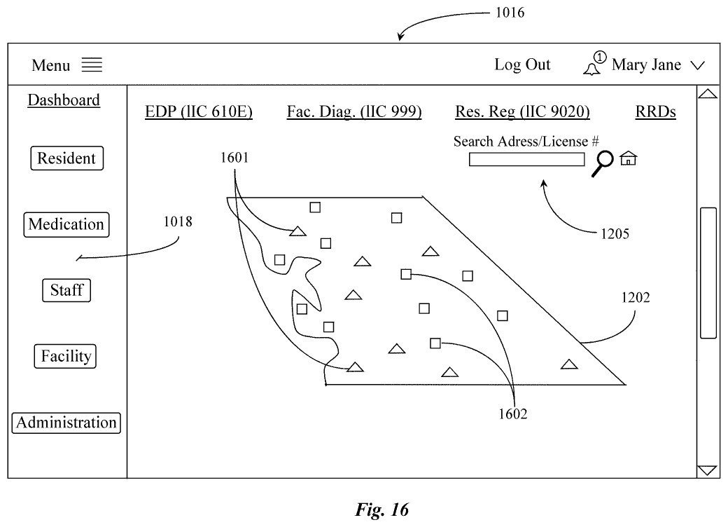

FIG. 16 is a screen shot of an ERT dashboard panel of SW 1016 of FIG. 1 depicting region 1202 of FIG. 12 customized to highlight temporary facilities and permanent facilities having vacancies. In this view, an administrator or operator or agent working to transfer residents from one facility to another has configured search return by using search utility 1205 to order only depicting of temporary facilities 1601 and permanent facilities 1602, where all of the depicted facilities have one or more than one vacancy. A temporary facility may be a shelter facility set up for an emergency causing the reason for the transfer. A permanent facility may be temporarily providing space for transferred residents. Any of facilities 1601 or 1602 may be selected and linked to for further browsing of information about the facility. The options in side bar 1018 may automatically link to the network hosted data relevant to any of the selected facilities, the data uploaded by the individual facilities and stored in data repository analogous to repository 1015 connected to portal server (ERTS) 1014 of FIG. 10.

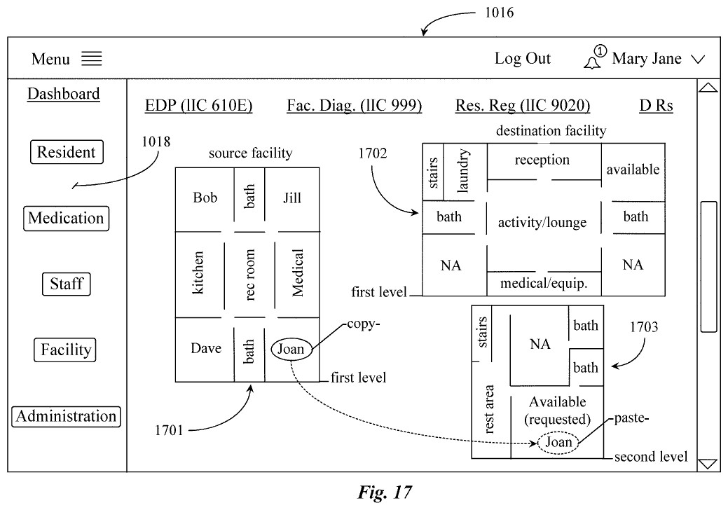

FIG. 17 is a screen shot of an ERT dashboard panel of SW 1016 of FIG. 1 depicting a source facility floor plan and a destination facility floor plan side by side. In this embodiment, an authorized individual working on transferring one or more residents has two facility floor plans called up and displayed on screen. A floor plan 1701 represents a source facility housing four residents Bob, Jill, Dave, and Joan. It is assumed in this example that Joan must be transferred to a new facility, but the remaining three residents are safe to keep as residents in the source facility.

Floor plan 1702 is a first level floor plan of a facility the administrator has determined is an adequate destination facility for resident Joan. The first level floor plan 1702 has two rooms not available (NA) and one room available, A second level floor plan of the facility 1702 represents a second story floor plan has one room that is not available and one room that is available. The administrator has determined to request a transfer for Joan if she can be placed in the available room on the second level floor plan of the facility.

In this embodiment, the source facility floor plan 1701 and the destination facility floor plan 1703 both first and second levels are interactive documents adapted to be manipulated as virtual objects. For example, the administrator may initiate a request to transfer Joan from her source facility 1702 by selecting and copying the icon or interactive text signifying Joan and then pasting the icon or copy of the text link into the desired available room, which in this case is on floor plan 1703 on the second level.

In one embodiment, the system will perform an automated comparability check (Joann's needs vs destination site assets and capabilities) and look for anything to show the administrator why the requested transfer of resident Joann may be rejected. If the system finds, for example, that the requirement of Joann to be on a dialysis machine and finds no dialysis machine registered with the destination facility signifying that there may not be one on site at the destination facility defined by floor plans 1702 and 1703. The system may cause a pop-up notification of a transfer caution based for example on a mismatch between Joann's requirement of the dialysis machine with the fact the machine is not part of the destination facility's on-site medical equipment.

The copy and paste operation the administrator may perform moving Joann from one facility room to another facility room launches a resident transfer request to the destination facility or agent representing the facility. In one embodiment, the service of the invention guarantees transfers based on matching needs (Joann) and assets/capabilities (destination facility).

In another embodiment an agent or administrator at the destination facility must review the request and approve or deny the transfer request. In one embodiment, an administrator may use attributes of Joann, specifically the critical need for a dialysis machine on site as a search query to have the system overlay the map with only facilities that have one or more available beds and that maintain at least one dialysis machine accessible to the residents. After the successful transfer of Joann, the source facility may update the change in data to the system server. The destination facility that accepted the transfer of Joann may now publish Joann's occupancy meta data for overlay on the map analogous to map 1200 of FIG. 12 relative to the facility icon of the destination facility.

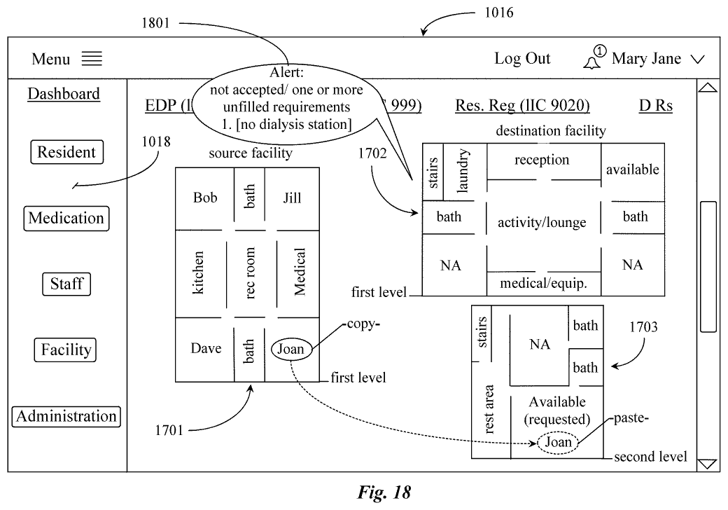

FIG. 18 is a screen shot of an ERT dashboard panel of SW 1016 of FIG. 1 depicting the facility floor plans of FIG. 17 with an alert overlay noting a resident transfer conflict with the destination facility. In this view, Joann has had a request for transfer initiated as described above in FIG. 17. In an embodiment where one or more destination facility assets, services, do not meet the needs of a resident, the system may generate a caution or alert in the form of a pop-up alert 1801 notifying the administrator of the issue. Alert 1801 informs the administrator that there is no dialysis station on site.

In the case of a successful transfer, a confirmation message may be sent to the administrator in the form of a notification, text message, on screen pop-up message or confirmation, email message, or a combination of those. The procedure of transfer of the resident and notification of transfer services may be scheduled by calendar automatically by the system or manually by the administrator. Some emergency situations may require fast action where residents must be evacuated from one or more facilities and then relocated among one or more facilities after evacuation to a shelter or ambulance or hospital has occurred. The administrator and staff may operate from remote locations and are not dependent on the lack of source facility data at the evacuated facility to practice the invention because the facility data remains hosted by the service.

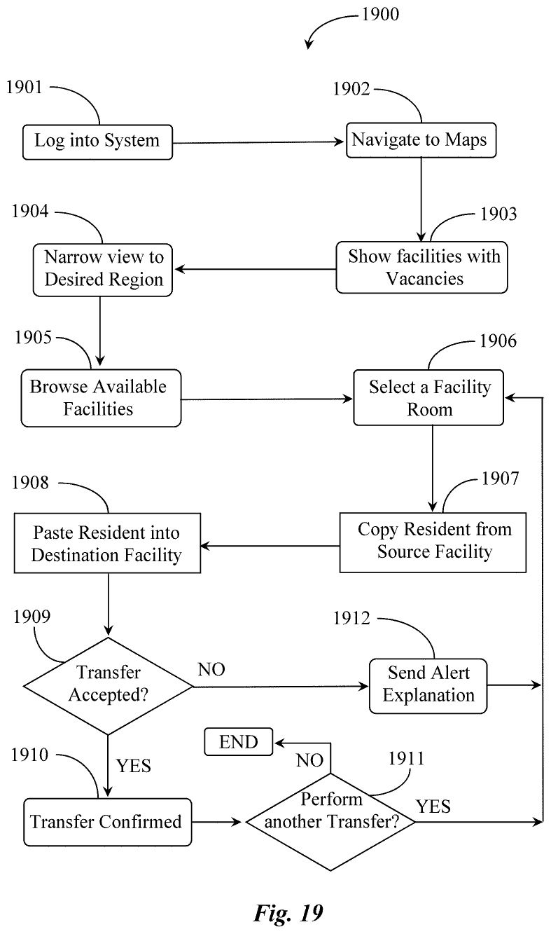

FIG. 19 is a process flow chart 1900 depicting steps for requesting at least one emergency resident transfer (ERT) using the system of the invention. At step 1901, an administrator or other authorized agent working for a care facility or multiple facilities may log into the system from any location on the network. In this process it is assumed the purpose of step 1900 is to request transfer for one or more than one resident from a source facility or facilities to a destination facility or facilities.

At step 1902, the administrator may navigate a dashboard panel to a state or regional digital map or maps depicting at least one overlay of icons representing facilities that may be considered potential destination facilities. In one embodiment, the facilities represented in the overlay on the map may be facilities that have current vacancies. In step 1903 those facilities may be represented as icons on a state or regional map.

Optionally in step 1904, the administrator may narrow the view to a region or portion of a map desired as a destination area for a transfer with the intention of selecting a facility or facilities in that desired region. At step 1905, the administrator may browse facilities by hovering a pointer (computer mouse, touch pad etc.) over the icons to get first level meta data about the facility. Selecting a facility may result in a network link to the facility wherein the dashboard data features are linked to the facility data held in a data repository hosted on the network.

At step 1906, the administrator may select a facility room after selecting a facility, the facility room desired as a destination for a resident to be transferred to. In this aspect, the administrator may also have an open list of residents for transfer or a floor plan of a source facility wherein the resident to be transferred is represented as an icon in a source room on that floor plan. The source plan may fit on screen along with the facility plan.

In the aspect where two plans are held side-by-side, the administrator may copy the representation of the resident (icon) from the source floor plan and paste the representation into the desired room of the destination facility at step 1908. In one aspect of the process, the administrator may also copy the resident's representation in the form of a hyperlink from a residents list. A transfer request of this nature is launched when the link is pasted into the desired room.

Notification of a resident transfer request action is live and linked to the destination facility via the destination floor plan and selected room. Both the system portal and the system end point or the destination facility are notified of the request. Moreover, the request may include all required data and documents, and pathways to the resident's plan, procedures, medical state and requirements, and proposed data of actual arrival of the resident to the destination facility.

At step 1909, the administrator may learn whether the transfer was accepted by the destination authority as an approved transfer. In one embodiment, transfers are approved if the needs and requirements of the resident are met by the destination facility. In one embodiment, transfers are peer reviewed by an agent or administrator working for the receiving facility and approved or rejected.

At step 1909, if the transfer is not accepted or there is a conflict, the system or peer reviewer may send an alert notification or screen pop-up to the requester at step 1912. The process may resolve back to step 1906 for the administrator to make another facility selection on the map or another room selection in the destination facility if the rejection was not facility wide.

At step 1909 if the transfer is accepted, the administrator may receive notification of confirmation via a screen pop, or other methods such as notification, email, text message, etc. at step 1910. At step 1911, the administrator may determine whether to transfer another resident or more residents. If the administrator is finished at step 1911, then the process may end at step 1913. If the administrator wishes to continue transferring residents, the process may loop back to step 1906 where the administrator may select new facilities and rooms.

It is noted herein that an administrator, in one embodiment, may copy or drag one or more representations of residents from a source floor plan or a source list of hyperlinks and then drop them over a facility with the same number of rooms. In this aspect, the system may compare the resident's attributes to the receiving facilities attributes and place the residents in the appropriate rooms based on their data and the data about each of the rooms. The administrator may then have an opportunity to review the suggested placement and agree to the logic of the system on behalf of the destination facility or not.

In one embodiment, confirmation of a transfer triggers an automated system process of records transfer and update, notification to transportation services of required transfer of the resident at a certain date of transfer. The facility objects dressed as icons in the map overlay are updated to show the new residency numbers.

It will be apparent with skill in the art that the emergency resident transfer system of the present invention may be provided using some or all the elements described herein. The arrangement of elements and functionality thereof relative to the emergency resident transfer network and system of the invention is described in different embodiments each of which is an implementation of the present invention.

While the uses and methods are described in enabling detail herein, it is to be noted that many alterations could be made in the details of the construction and the arrangement of the elements without departing from the spirit and scope of this invention. The present invention is limited only by the breadth of the claims below.

1. An interactive user interface (UI) accessible on a computer network through a connected computing device, the UI enabling request for transfer and initiation of transfer of a resident in a source residential facility connected to the network to a destination residential facility connected to the network comprising:
coded instruction residing on a non-transitory medium coupled to a server connected to the network, the server coupled to at least one data repository, the coded instruction commanding the server to;
(a) receive data from residential facilities connected to the network;
(b) store the data received in (a) in updated state in the at least one data repository;
(c) serve at least one digital instance of the UI to at least one authority having charge over at least one of the residential facilities upon request of the at least one authority;
(d) serve at least one digital map, the digital map overlaid with interactive indicia geographically and texturally identifying the connected facilities to the at least one authority of (c) upon request;
(e) receive a request for transfer of one or more residents from the one or more connected source facilities to one or more connected destination facilities;
(f) run a comparative algorithm between data stored for the one or more source facilities and data stored for one or more of the destination facilities of the request received in (e);
(g) based on the data comparison of (f) alert or do not alert the requesting authority or authorities of a potential conflict with the desired goal or goals of the requested transfer of (e);
(h) serve notification of transfer request, transfer schedule and required documents to one or more destination authorities for review and response;
(i) upon destination authority(s) acceptance as a response, confirm via server notification to the requesting authority or authorities, successful transfer of the one or more residents, or upon authority rejection as a response, reject via server notification to the requesting authority or authorities transfer of the one or more residents; and
(j) updating map meta data associated with the interactive indicia overlaid on the digital map.

2. The UI of claim 1, wherein the network is the Internet and any connected sub networks.

3. The UI of claim 2, wherein in (a) the received data is uploaded from the facilities over the Internet.

4. The UI of claim 1, wherein the residential facilities include permanent resident residential facilities and temporary resident residential facilities.

5. The UI of claim 1, wherein the residential facilities include residential care homes defined by converted houses and residential care facilities defined by converted commercial buildings.

6. The UI of claim 1, wherein the UI includes multiple interactive options for linking to and viewing categorically diverse data sets associated with a facility.

7. The UI of claim 6, wherein the diverse data sets include data about the facility physical capacities, data about facility staff, data about facility residents, and data about facility emergency and safety planning.

8. The UI of claim 1, wherein the connected device is a mobile telephony device having connection to the network in a physical location outside of a facility.

10. The UI of claim 1, wherein the data received in (a) includes a list of current residents and the current and scheduled number of vacancies if any at the facilities.

11. The UI of claim 1, wherein the data received in (a) includes one or more digital floor plans the floor plans overlaid with interactive indicia geographically and texturally identifying at least occupied and non-occupied or otherwise vacant living spaces.

12. The UI of claim 1, wherein the interactive indicia geographically and texturally identifying the connected facilities in the digital map of (d) contain links to the parent facility data when selected by the one or more authorities in the digital map, the multiple data options in the UI automatically linked to those data sets upon selection.

13. The UI of claim 12, wherein at least one of the multiple data options enables view and interaction with the one or more digital floor plans of claim 11.

14. The UI of claim 13, wherein interaction with the one or more digital floor plans of claim 11 include computer-aided copying and pasting one or more objects representing residents needing transfer onto the vacant rooms or beds of the one or more floor plans, or computer-aided dragging and dropping one or more objects representing residents needing transfer onto the vacant rooms or beds of the one or more floor plans.

15. The UI of claim 14, wherein the computer interaction methods copy and paste and drag and drop as stated in claim 14 comprise the one or more requests for transfer in (e).
